(19) United States
(12) Patent Application Publication
(10) Pub. No.: US 2021/0121079 A1
(43) Pub. Date: Apr. 29, 2021

(54) APPARATUS FOR MEASURING HEMODYNAMIC PARAMETERS

(71) Applicant: NIHON KOHDEN CORPORATION, Tokyo (JP)

(72) Inventors: Yoshihiro SUGO, Tokorozawa-shi (JP); Yoshiharu HARADA, Tokorozawa-shi (JP); Mami SAKAI, Tokorozawa-shi (JP)

(21) Appl. No.: 17/070,259

(22) Filed: Oct. 14, 2020

(30) Foreign Application Priority Data
Oct. 24, 2019 (JP) 2019-193426

(51) Int. Cl. A61B 5/02; A61B 5/0402; A61B 5/021

(52) U.S. Cl. CPC A61B 5/02028; A61B 5/02108; A61B 5/0402

(57) ABSTRACT

An apparatus for measuring hemodynamic parameters that can measure information relating to a cardiac contraction function of a subject with a higher accuracy and with a light burden of the subject is provided. An apparatus for measuring hemodynamic parameters includes: an electrocardiogram acquiring interface that acquires an electrocardiogram of a subject; a pulse wave acquiring interface that acquires a pulse wave of the cranio-cervical region of the subject; and a calculator that calculates information relating to cardiac functions of the subject, based on a pulse wave transit time obtained from the electrocardiogram and the pulse wave.

### CROSS-REFERENCE TO RELATED APPLICATIONS

The present application claims priority from Japanese Patent Application No. 2019-193426, filed Oct. 24, 2019, the entire content of which is incorporated herein by reference.

The presently disclosed subject matter relates an apparatus for measuring hemodynamic parameters.

### BACKGROUND

U.S. Patent Publication No. 2018/0263503 discloses an apparatus for measuring hemodynamic parameters including: an electrocardiogram acquiring section that acquires an electrocardiogram of a subject; a pulse wave acquiring section that acquires a pulse wave of an upper arm of the subject; and a calculator that calculates information relating to a cardiac contraction function and the like of the subject, based on a pulse wave transit time obtained from the electrocardiogram and the pulse wave.

As a method for measuring a pulse wave of an upper arm of a subject, there is a method in which a cuff is attached to, for example, an upper arm of a subject, the artery of the subject and the periphery of the artery are pressed, and then the pulse wave of the upper arm is measured. In the method, however, the motion of the subject to whom the cuff is attached is restrained during measurement of the pulse wave. Therefore, the apparatus for measuring hemodynamic parameters disclosed in U.S. Patent Publication No. 2018/0263503 has room for improvement.

### SUMMARY

The presently disclosed subject matter is provided with an apparatus for measuring hemodynamic parameters that can measure information relating to a cardiac contraction function of a subject with a higher accuracy and with a light burden of the subject.

A first aspect of apparatus for measuring hemodynamic parameters includes:

an electrocardiogram acquiring section that acquires an electrocardiogram of a subject;

a pulse wave acquiring section that acquires a pulse wave of a cranio-cervical region of the subject; and

a calculator that calculates information relating to cardiac functions of the subject, based on a pulse wave transit time obtained from the electrocardiogram and the pulse wave.

A second aspect of apparatus for measuring hemodynamic parameters includes:

an electrocardiogram acquiring section that acquires an electrocardiogram of a subject;

a first pulse wave acquiring section that acquires a first pulse wave in a cranio-cervical region of the subject;

a second pulse wave acquiring section that acquires a second pulse wave in at least one of extremities of the subject;

a first calculator that calculates a first pulse wave transit time from the electrocardiogram and the first pulse wave; and

a second calculator that calculates a second pulse wave transit time from the electrocardiogram and the second pulse wave.

### BRIEF DESCRIPTION OF DRAWINGS

FIG. 1 is a block diagram exemplifying the configuration of an apparatus for measuring hemodynamic parameters of a first embodiment.

FIG. 2 is a block diagram exemplifying the configuration of a controller of the apparatus for measuring hemodynamic parameters.

FIG. 3 is a flowchart exemplifying the operation of the apparatus for measuring hemodynamic parameters.

FIG. 4 exemplarily illustrates an electrocardiogram, the aortic root blood pressure, the earlobe pulse wave, the brachial artery pulse wave, and the pulse waves in the extremities.

FIG. 5 illustrates an example of a display image on the screen.

FIG. 6 is a block diagram exemplifying the configuration of an apparatus for measuring hemodynamic parameters of a second embodiment.

FIG. 7 is a flowchart exemplifying the operation of the apparatus for measuring hemodynamic parameters.

FIG. 8 illustrates another example of the display image on the screen.

FIG. 9 illustrates a further example of the display image on the screen.

FIG. 10 illustrates a still further example of the display image on the screen.

FIG. 11 illustrates a still further example of the display image on the screen.

### DETAILED DESCRIPTION

Hereinafter, exemplary embodiments will be described in detail with reference to the accompanying drawings.

### First Embodiment

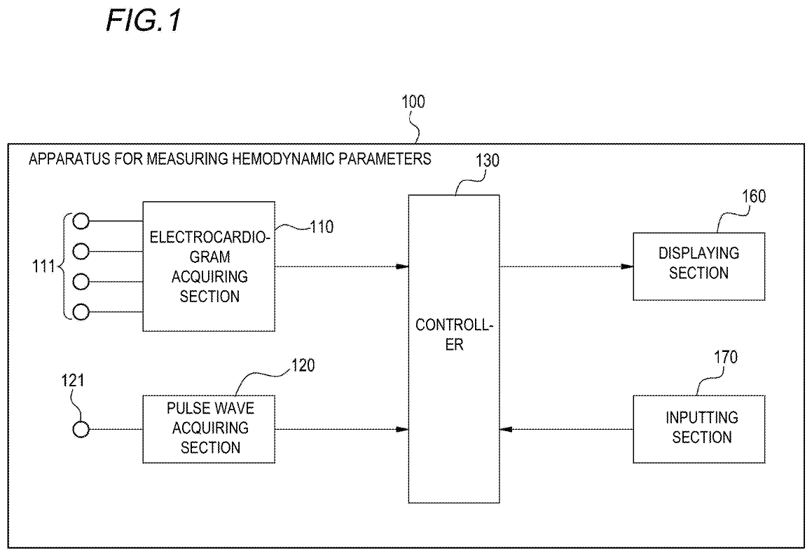

FIG. 1 is a block diagram exemplifying the configuration of an apparatus for measuring hemodynamic parameters 100 of a first embodiment. The apparatus 100 may include an electrocardiogram acquiring section 110, a pulse wave acquiring section 120, a controller 130, a displaying section 160, and an inputting section 170.

The electrocardiogram acquiring section 110 continuously detects an electrocardiogram showing the action potential that is produced by excitation in the myocardium, through a plurality of electrodes 111 attached to predetermined portions of the subject. The electrocardiogram acquiring section 110 transmits the detected electrocardiogram to the controller 130. The electrocardiogram acquiring section 110 may be an interface between the plurality of electrodes 111 and the controller 130. One of example of the interface may be a connector configured to wiredly or wirelessly connect each other.

The pulse wave acquiring section 120 continuously detects a pulse wave through a photoplethysmography sensor 121. For example, the sensor 121 is a sensor of the continuous measurement type. The sensor 121 may include a light emitter and a light detector. For example, the photoplethysmography sensor 121 may be an ear clip type sensor that nips the earlobe of the subject to be attached to the subject, or a sensor that is applied to the forehead, neck, or the like of the subject to be attached to the subject. In this way, the photoplethysmography sensor 121 is attached to, for example, the earlobe, the forehead, or the carotid artery in order to detect the pulse wave of the cranio-cervical region of the subject. In the case where the photoplethysmography sensor 121 is an ear clip type sensor, for example, the sensor 121 is attached to the subject so as to nip the earlobe. In this case, the photoplethysmography sensor 121 emits a light beam toward the earlobe that is the attachment portion. The photoplethysmography sensor 121 receives a reflected or transmitted light beam of the emitted light beam. The sensor 121 transmits information relating to the reflected or transmitted light beam that is received, to the pulse wave acquiring section 120. The pulse wave acquiring section 120 acquires the cranio-cervical pulse wave that is the pulse wave in the cranio-cervical region, based on the information relating to the reflected or transmitted light beam that is received. The pulse wave acquiring section 120 transmits the acquired cranio-cervical pulse wave to the controller 130. The pulse wave acquiring section 120 may be an interface between the photoplethysmography sensor 121 and the controller 130. One of example of the interface may be a connector configured to wiredly or wirelessly connect each other.

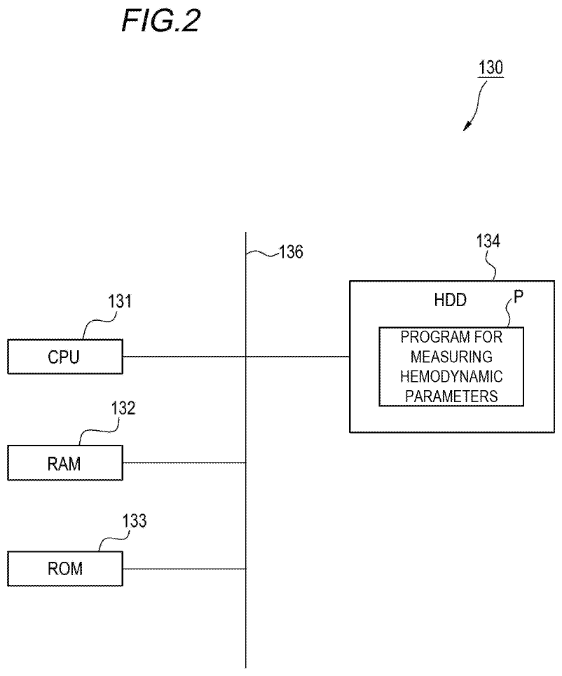

FIG. 2 is a block diagram illustrating a configuration example of the controller 130 of the apparatus for measuring hemodynamic parameters 100.

The controller 130 may include at least one Central Processing Unit (CPU) 131, at least one Random Access Memory (RAM) 132, at least Read Only Memory (ROM) 133, and an Hard Disk Drive (HDD) 134. These components are connected to one another in a mutually communicable manner by a bus 136. The CPU 131 can function as a calculator and a display image producing section.

In accordance with programs, the CPU 131 controls the components of the controller 130, the electrocardiogram acquiring section 110, and the pulse wave acquiring section 120, and performs various calculations. The CPU 131 can control the displaying section 160 and the inputting section 170. The CPU 131 can produce display image data for displaying information relating to the calculated cardiac functions. The CPU 131 can cause a display image that is based on the information relating to the cardiac functions of the subject and the display image data, to be displayed on the displaying section 160.

The RAM 132 is a volatile storage device, and can temporarily store programs and various data including measurement data.

The ROM 133 is a non-volatile storage device, and can store a wide variety of data including various set data that are used when a program for measuring hemodynamic parameters P is executed.

The HDD 134 stores various programs including the operating system and the program for measuring hemodynamic parameters P, and a wide variety of data including measurement data and basic information of the subject. The basic information of the subject can include the ID, name, and age of the subject.

Returning to FIG. 1, the displaying section 160 and inputting section 170 that are provided to the apparatus 100 will be described.

The displaying section 160 can display the information that is received from the controller 130, and that relates to the cardiac functions of the subject, the display image, etc. The displaying section 160 can be configured by, for example, a liquid crystal display.

The inputting section 170 receives various inputs. For example, the various inputs can include an input of the basic information of the subject. The inputting section 170 can be configured by a keyboard, a touch panel, or dedicated keys.

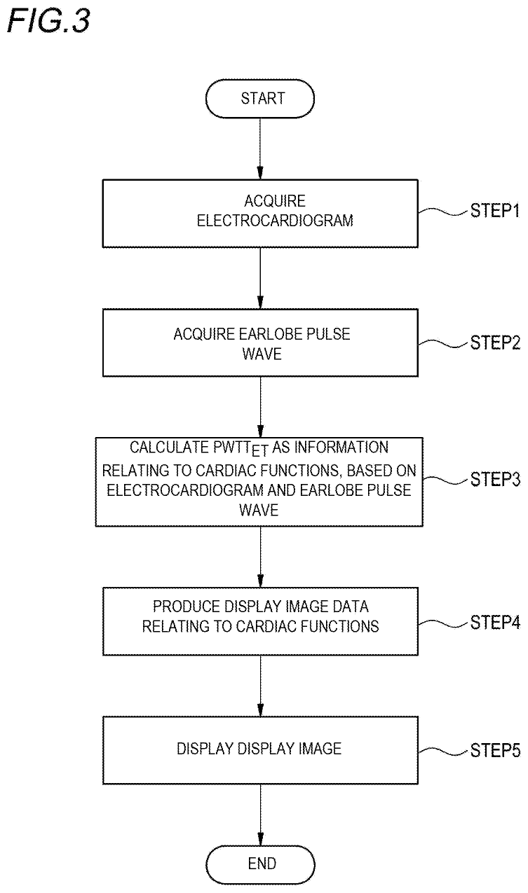

FIG. 3 is a flowchart illustrating the operation of the apparatus 100. The flowchart is executed by the CPU 131 in accordance with the program for measuring hemodynamic parameters P.

The CPU 131 controls the electrocardiogram acquiring section 110 to acquire an electrocardiogram of the subject (STEP 1).

The CPU 131 controls the pulse wave acquiring section 120 to acquire the earlobe pulse wave (an example of the cranio-cervical pulse wave) of the subject (STEP 2).

Next, the CPU 131 calculates the pulse wave transit time (hereinafter, referred to as “PWTT”) from the electrocardiogram and the earlobe pulse wave. Specifically, the CPU 131 acquires an electrocardiogram from the electrocardiogram acquiring section 110. The CPU 131 further acquires the earlobe pulse wave that is the pulse wave of the earlobe, based on the information relating to the reflected or transmitted light beam that is received by the photoplethysmography sensor 121 attached to the subject. Then, the CPU 131 calculates the PWTT that is calculated from the electrocardiogram and the earlobe pulse wave (hereinafter, such a PWTT is referred to as “PWTT_{ET}”), as information relating to the cardiac functions (STEP 3).

As described later, the PWTT_{ET} particularly reflects the cardiac contraction function in the cardiac functions. The information relating to the cardiac functions can include information relating to the all cardiac functions in addition to the cardiac contraction function. For example, the information relating to the cardiac functions includes the PWTT_{ET}, a value (PWTT_{ET}/ET) that will be described later, and that is obtained by dividing the PWTT_{ET} by the ejection time (hereinafter, referred to as “ET”), and changes over time in these values.

The reason that the PWTT_{ET} reflects the cardiac contraction function will be described.

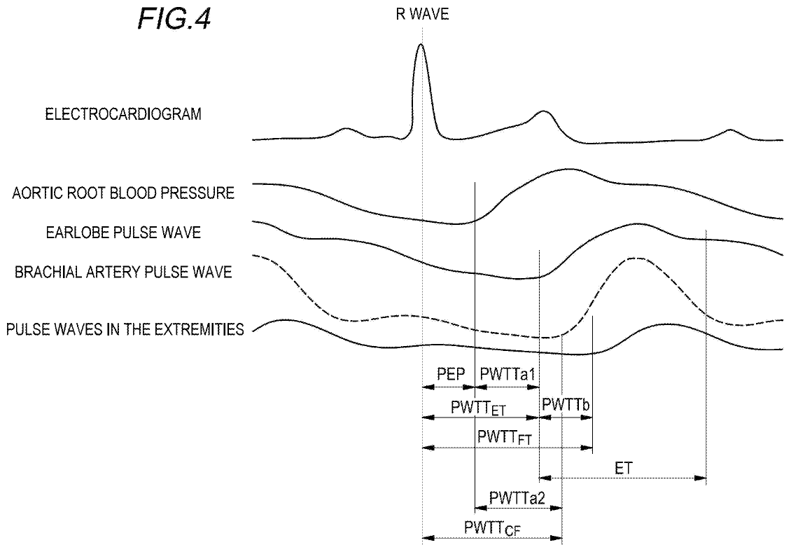

FIG. 4 exemplarily illustrates the electrocardiogram, the aortic root blood pressure, the earlobe pulse wave, the brachial artery pulse wave, and the pulse waves in the extremities. The pulse waves in the extremities is the fingertip pulse wave that is acquired from at least one of the extremities (the fingertip of the right hand, that of the left hand, the toe tip of the right foot, and that of the left foot).

Referring to FIG. 4, a PEP, the PWTT, and the ET will be described. The PEP is a time period from the start of the ventricular contraction to the beginning of the actual blood ejection. The PEP appears as the time period from the R wave in the electrocardiogram to the start of rising of the aortic root blood pressure. The PEP reflects the cardiac contraction function. This is because the left ventricular ejection fraction (hereinafter the ejection fraction is referred to as “EF”) that is one of the indexes relating to the contraction function of the left ventricle has a negative correlation with the PEP. The EF is a value that is obtained by dividing the amount of blood (ejection amount) that is ejected per beat from the heart, by the left ventricular volume at heart dilatation.

Commonly, the aortic root blood pressure is measured by a catheter in which a pressure sensor is disposed in the tip end. Therefore, it is difficult to non-invasively measure the PEP.

The PWTT_{ET} is a time period from the R wave in the electrocardiogram to the start of rising of the earlobe pulse wave, and the sum of the PEP and the PWTT that is required for the pulse wave to reach from the heart through the artery to the earlobe (hereinafter, such a PWTT is referred to as “PWTTa1”). The PWTT_{ET} can be non-invasively measured based on the electrocardiogram and the earlobe pulse wave.

Next, a PWTT_{CF} will be described. The broken line in FIG. 4 is a graph relating to the brachial artery pulse wave. For example, the brachial artery pulse wave is acquired through a cuff that is attached to the upper arm of a subject. The PWTT_{CF} is a PWTT that is calculated from the electrocardiogram and the brachial artery pulse wave, and a time period from the R wave in the electrocardiogram to the start of rising of the brachial artery pulse wave. The PWTT_{CF} is the sum of the PEP and the PWTT that is required for the pulse wave to reach from the heart through the artery to the upper arm to which the cuff is attached (hereinafter, such a PWTT is referred to as “PWTTa2”).

The earlobe is at a position that is more proximal to the heart as compared with, for example, the upper arm. Therefore, the time length of the PWTTa1 is shorter than that of the PWTTa2. Namely, the proportion of the PEP that occupies the PWTT_{ET} is larger than that of the PEP that occupies the PWTT_{CF}. Therefore, the influence exerted on the PWTT_{ET} by the PWTTa1 that depends on the blood pressure is smaller in degree than that exerted on the PWTT_{CF} by the PWTTa2 that depends on the blood pressure. Consequently, the PWTT_{ET} reflects the cardiac contraction function more accurately than the PWTT_{CF}. Therefore, the PWTT_{ET} can be used as information relating to the cardiac functions, in place of the PEP and the PWTT_{CF}.

Also a value (PEP/ET) that is obtained by dividing the PEP by the ET correlates with the EF, and hence reflects the cardiac contraction function. The ET is a time period from rising of the earlobe pulse wave to a notch. As described above, the PWTT_{ET} can be used in place of the PEP and the PWTT_{CF}. Therefore, also a value (PWTT_{ET}/ET) that is obtained by dividing the PWTT_{ET} by the ET can be used in place of the value (PEP/ET) that is obtained by dividing the PEP by the ET, and a value (PWTT_{CF}/ET) that is obtained by dividing the PWTT_{CF} by the ET. Consequently, the value (PWTT_{ET}/ET) that is obtained by dividing the PWTT_{ET} by the ET can be used as information relating to the cardiac functions. The ET can be measured based on the earlobe pulse wave.

The PWTT that is required for the pulse wave to reach from the earlobe to which the photoplethysmography sensor 121 is attached, through the peripheral artery to one of the extremities (hereinafter, such a PWTT is referred to as “PWTTb”) will be described later in the description of a second embodiment.

The CPU 131 calculates the PWTT_{ET} and the ET based on the earlobe pulse wave that is acquired from the pulse wave acquiring section 120. Therefore, the PWTT_{ET} and the value (PWTT_{ET}/ET) that is obtained by dividing the PWTT_{ET} by the ET can be acquired in the process for measuring the earlobe pulse wave by the photoplethysmography sensor 121 attached to the earlobe of the subject. Namely, the CPU 131 can calculate information relating to the cardiac functions in association with the pulse wave measurement.

The CPU 131 may calculate a change over time in the PWTT_{ET} with respect to the PWTT_{ET} that is calculated from the earlobe pulse wave at a predetermined timing, as information relating to the cardiac functions. For example, the timing of hospital discharge can be set as the predetermined timing. This enables information relating to the cardiac functions to be used as a continuous index of the cardiac functions in a time when the subject is at home.

Returning to FIG. 3, STEP 4 and subsequent steps will be described. The CPU 131 produces display image data relating to the cardiac functions in which, for example, the acquisition time when the electrocardiogram and the earlobe pulse wave are acquired is displayed together with the PWTT_{ET} obtained from the electrocardiogram and earlobe pulse wave that are acquired at the acquisition time (STEP 4).

In this case, the CPU 131 produces display image data in which information relating to the cardiac functions is displayed in the form of two-dimensional graphs in which the acquisition time when the electrocardiogram and the earlobe pulse wave are acquired is set as the abscissa, and the PWTT_{ET} that is obtained from the electrocardiogram and earlobe pulse wave that are acquired at the acquisition time of the abscissa is set as the ordinate.

The CPU 131 sends the produced display image data to the displaying section 160, and causes the displaying section 160 to display a display image based on the display image data (STEP 5).

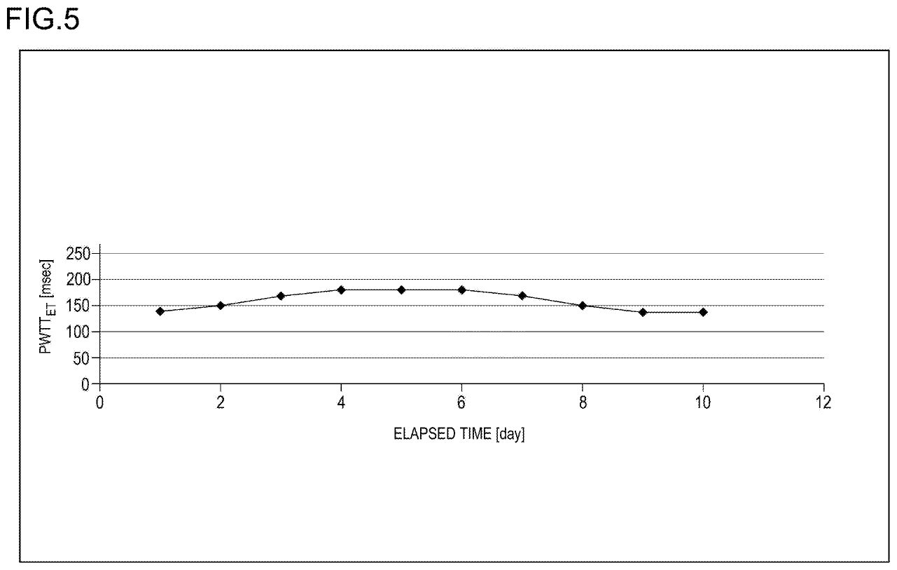

FIG. 5 illustrates an example of the display image in which the PWTT_{ET} is displayed, and which relates to the cardiac functions.

The display image of FIG. 5 is configured by two-dimensional graphs of time transitions of the PWTT_{ET} that is information relating to the cardiac functions, and illustrates changes over time in these values. The abscissa indicates the elapsed time from the timing of hospital discharge of the subject. The display of the PWTT_{ET} reflecting the cardiac contraction function enables a sign of heart failure to be early detected.

Although, in the display image illustrated in FIG. 5, the abscissa indicates the elapsed time from the timing of hospital discharge of the subject, the abscissa may indicate the acquisition time when the electrocardiogram and the earlobe pulse wave are acquired. The acquisition time may be the year, month, date, and time (hour and minute) when the electrocardiogram and the earlobe pulse wave are acquired. In this case, the ordinate may indicate the PWTT_{ET} that is obtained from the electrocardiogram and earlobe pulse wave which are acquired at the acquisition time of the abscissa. The acquisition time may be additionally indicated at each plot of the two-dimensional graphs illustrated in FIG. 5.

The embodiment attains the following effects.

The CPU 131 calculates information relating to the cardiac functions of the subject based on the pulse wave transit time obtained from the electrocardiogram and the earlobe pulse wave (cranio-cervical pulse wave). The earlobe pulse wave can be measured more accurately than the brachial artery pulse wave, and more correctly reflects the central information. According to the configuration as described above, therefore, information relating to the cardiac contraction function of the subject can be measured with a higher accuracy and with a light burden of the subject.

Moreover, the earlobe pulse wave is acquired by the photoplethysmography sensor 121. The brachial artery pulse wave is acquired by measurement while attaching a cuff to the upper arm of the subject. By contrast, the earlobe pulse wave is acquired by nipping the earlobe with the sensor 121. That is, the burden on the subject can be made smaller as compared to the case where the pulse wave is measured by using a cuff. Moreover, the pulse wave of the subject can be continuously measured without restraining the motion of the subject as compared with the case where the pulse wave is measured by using a cuff.

According to the thus configured apparatus for measuring hemodynamic parameters, information relating to the cardiac functions of the subject is calculated based on the pulse wave transit time that is obtained from the electrocardiogram and the pulse wave of the cranio-cervical region. The pulse wave of the cranio-cervical region can be measured more accurately than that of the upper arm, and more correctly reflects the central information. As compared with the measurement of the pulse wave of the upper arm by using a cuff, moreover, the measurement of the pulse wave of the cranio-cervical region can be performed more simply.

According to the configuration, therefore, information relating to the cardiac contraction function of the subject can be measured with a higher accuracy and with a light burden of the subject.

### Second Embodiment

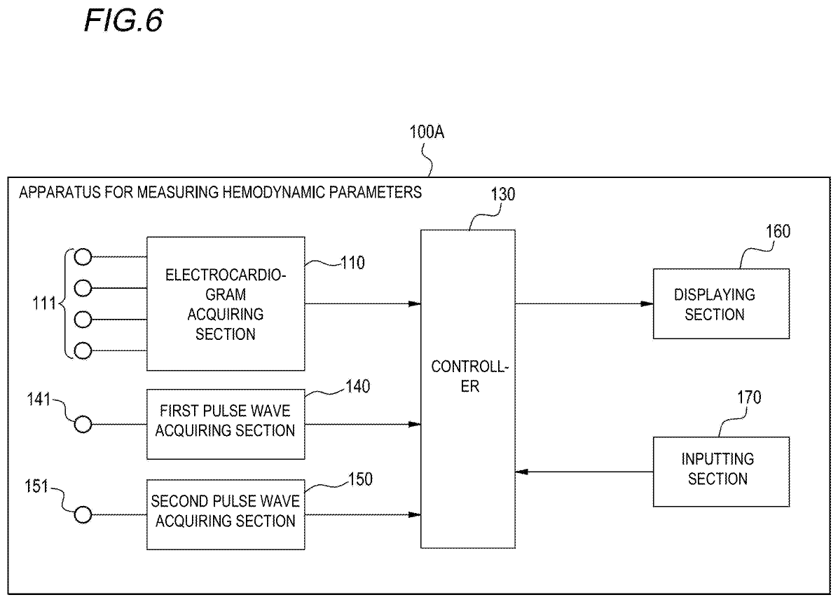

Next, the second embodiment will be described in detail with reference to FIGS. 6 to 11. The embodiment is different from the first embodiment in the following points. In the embodiment, a first pulse wave transit time is calculated based on the electrocardiogram of the subject, and a first pulse wave in the earlobe, and a second pulse wave transit time is calculated based on the electrocardiogram and a second pulse wave in the extremities. Moreover, a display screen on which the first pulse wave transit time and the second pulse wave transit time are simultaneously displayed is produced. In the description of the embodiment, description which is duplicated with that of the first embodiment will be omitted or simplified.

FIG. 6 is a block diagram exemplifying the configuration of an apparatus for measuring hemodynamic parameters 100A of the second embodiment. As exemplified in FIG. 6, the apparatus 100A is different from the apparatus for measuring hemodynamic parameters 100 in that the apparatus 100A further includes a second pulse wave acquiring section 150 and a photoplethysmography sensor 151. A first pulse wave acquiring section 140 is configured in the same or similar manner as the pulse wave acquiring section 120 in the first embodiment, and a photoelectric sensor 141 is configured in the same or similar manner as the photoplethysmography sensor 121 in the first embodiment.

The second pulse wave acquiring section 150 continuously detects a pulse wave through the photoplethysmography sensor 151. The sensor 151 may be configured in the same or similar manner as the photoplethysmography sensor 121. For example, the photoplethysmography sensor 151 is attached to at least one of the extremities (the fingertip of the right hand, that of the left hand, the toe tip of the right foot, and that of the left foot) of the subject. The second pulse wave acquiring section 150 acquires one of the pulse waves in the extremities that is the pulse wave of one of the extremities to which the photoplethysmography sensor 151 is attached, based on information relating to the reflected or transmitted light beam that is received by the sensor 151. The pulse waves in the extremities can constitute the second pulse wave.

In the second embodiment, the CPU 131 can function as the first calculator, the second calculator, and the display image producing section.

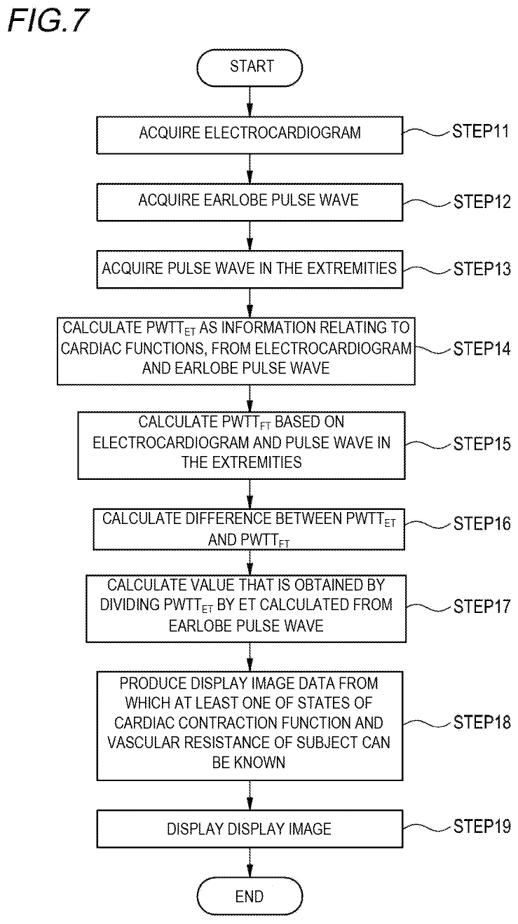

FIG. 7 is a flowchart illustrating the operation of the apparatus 100A. The flowchart can be executed by the CPU 131 in accordance with the program for measuring hemodynamic parameters P.

STEPS 11 and 12 are same as or similar to STEPS 1 and 2 of FIG. 3, and therefore their description is omitted.

The second pulse wave acquiring section 150 acquires one of the pulse waves in the extremities from the photoplethysmography sensor 151 that is attached to at least one of the extremities (STEP 13).

STEP 14 is same as or similar to STEP 3 of FIG. 3, and therefore its description is omitted.

Based on the electrocardiogram and one of the pulse waves in the extremities that is acquired from one of the extremities to which the photoplethysmography sensor 151 is attached, the CPU 131 calculates the PWTT that is required for the pulse wave to reach from the heart through the artery to one of the extremities to which the sensor 151 is attached (hereinafter, such a PWTT is referred to as “PWTT_{FT}”, see FIG. 4) (STEP 15). Specifically, the CPU 131 acquires one of the pulse waves in the extremities from the photoplethysmography sensor 151, and calculates the PWTT_{FT} based on the electrocardiogram and one of the pulse waves in the extremities. The PWTT_{FT} constitutes the second pulse wave transit time.

Referring again to FIG. 4, the relationship between the PWTT_{ET} and the PWTT_{FT} will be described. The PWTT_{FT} is the time period from the R wave in the electrocardiogram to the start of rising of one of the pulse waves in the extremities, and is the sum of the PEP, the PWTTa1, and the PWTTb. The PWTTb is the difference (PWTT_{FT}-PWTT_{ET}) between the PWTT_{FT} and the PWTT_{ET}.

When the PWTT_{ET} and the PWTT_{FT} are compared with each other, the PWTT_{FT} contains the PWTTb. and by contrast the PWTT_{ET} does not contain the PWTTb. The PWTTb is the PWTT that is required for the pulse wave to reach from the earlobe to which the photoelectric sensor is attached, through the peripheral artery to the fingertip, and reflects the vascular resistance. Therefore, the state of the vascular resistance can be known by comparing the PWTT_{ET} with the PWTT_{FT} with each other. On the other hand, the PWTT_{ET} does not contain the PWTTb, and hence the proportion of the PEP that occupies the PWTT_{ET} is larger than that of the PEP that occupies the PWTT_{FT}. As described above, the PEP reflects the cardiac contraction function. When the PWTT_{FT} and the PWTT_{ET} are compared with each other, therefore, also the state of the cardiac contraction function can be known.

Returning to FIG. 7, STEP 16 and subsequent steps will be described. The CPU 131 calculates the difference (i.e., the PWTTb) between the PWTT_{ET} and the PWTT_{FT}. Moreover, the CPU 131 calculates two-dimensional graphs of the relationship between any two of the PWTT_{ET}, the PWTT_{FT}, and the difference between the PWTT_{ET} and the PWTT_{FT}.

The CPU 131 calculates the value that is obtained by dividing the PWTT_{ET} by the ET which is calculated from the earlobe pulse wave (STEP 17).

The CPU 131 produces display image data from which at least one of the states of the cardiac contraction function and vascular resistance of the subject can be known (STEP 18).

The display image data may include two-dimensional graphs of at least two of the relations of the PWTT_{FT}, the PWTT_{FT}, and the difference between the PWTT_{ET} and the PWTT_{FT}.

The display image data can include at least two of the PWTT_{ET}, the PWTT_{FT}, and the difference between the PWTT_{ET} and the PWTT_{ET}. In a display image based on such display image data, the acquisition time when the electrocardiogram, the earlobe pulse wave, and the pulse waves in the extremities are acquired can be further displayed. In the display image, namely, at least two of the PWTT_{ET} that is acquired from the electrocardiogram and earlobe pulse wave which are acquired at the acquisition time, the PWTT_{FT} that is calculated from the electrocardiogram and one of the pulse waves in the extremities, and the difference between the two values can be displayed together with the acquisition time.

The display image data can include two-dimensional graphs in which the acquisition time is set as the abscissa, and at least two of the PWTT_{ET} calculated from the electrocardiogram and earlobe pulse wave that are acquired at the acquisition time of the abscissa, the PWTT_{FT} calculated from the electrocardiogram and one of the pulse waves in the extremities, and the difference between the two values are set as the ordinates.

The display image data can be display image data relating to a display image in which at least one of the PWTT_{ET}, the PWTT_{FT}, and the difference between the PWTT_{ET} and the PWTT_{FT} is displayed. The display image data can be display image data relating to a display image of two-dimensional graphs of the relationship between at least two of the PWTT_{ET}, the PWTT_{FT}, and the difference between the PWTT_{ET} and the PWTT_{FT}.

The display image can further include the value (PWTT_{ET}/ET) that is obtained by dividing the PWTT_{ET} by the ET.

The CPU 131 sends the produced display image data to the displaying section 160, and causes the displaying section 160 to display a display image based on the display image data (STEP 19).

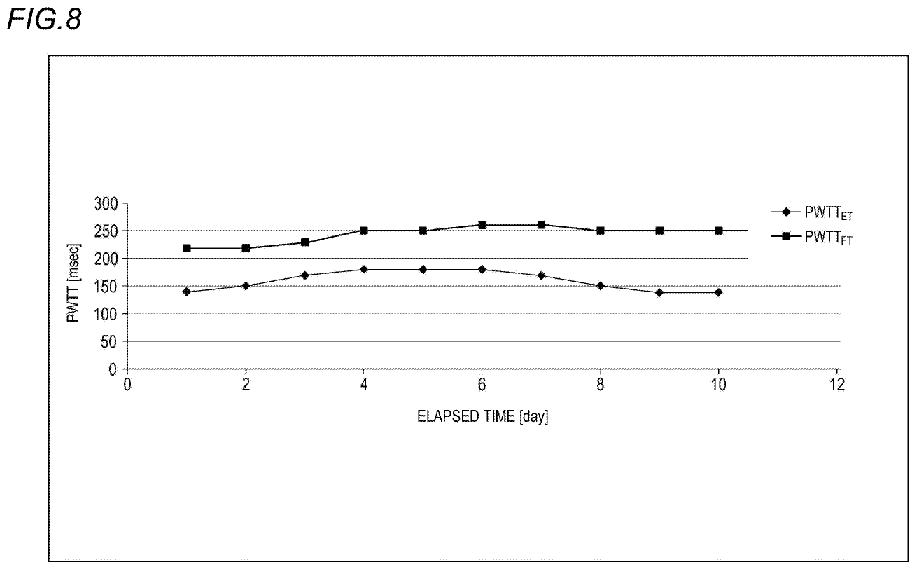

FIG. 8 illustrates an example of a display image in which the PWTT_{ET} and the PWTT_{FT} are simultaneously displayed.

The display image of FIG. 8 is configured by two-dimensional graphs of time transitions of the PWTT_{ET} and the PWTT_{FT}, and illustrates changes over time in these values. The abscissa indicates the elapsed time from the timing of hospital discharge of the subject. The display of the PWTT_{ET} reflecting the cardiac contraction function in the display image enables a sign of heart failure to be early detected.

Although, in the display image illustrated in FIG. 8, the abscissa indicates the elapsed time from the timing of hospital discharge of the subject, the abscissa may indicate the acquisition time when the electrocardiogram, the earlobe pulse wave, and the pulse waves in the extremities are acquired. In this case, the ordinates indicate the PWTT_{ET} and PWTT_{FT} that are obtained from the electrocardiogram, earlobe pulse wave, and pulse waves in the extremities which are acquired at the acquisition time of the abscissa. The acquisition time may be additionally indicated at each plot of the two-dimensional graphs illustrated in FIG. 8.

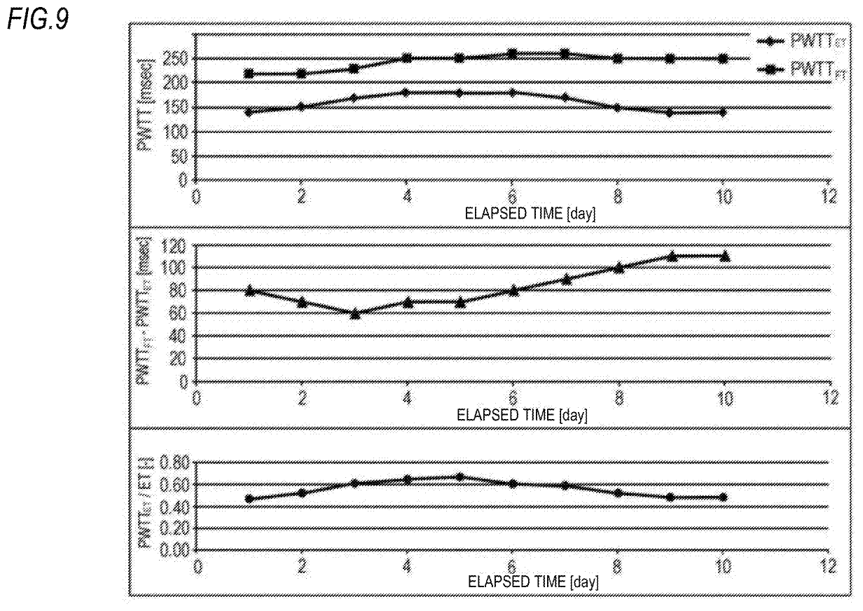

FIG. 9 illustrates an example of a display image in which the PWTT_{ET}, the PWTT_{FT}, the difference between the PWTT_{ET} and the PWTT_{FT}, and a value which is obtained by dividing the PWTT_{ET} by the ET are simultaneously displayed. The display image is configured by two-dimensional graphs of time transitions of the PWTT_{ET}, the PWTT_{FT}, the difference between the PWTT_{ET} and the PWTT_{FT}, and the value which is obtained by dividing the PWTT_{ET} by the ET, and illustrates changes over time in these values. The abscissa indicates the elapsed time from the timing of hospital discharge of the subject. In the display image, the span and scale of the elapsed time of the abscissa are common in the two-dimensional graph of the PWTT_{ET} and the PWTT_{FT}, that of the difference between the PWTT_{FT} and the PWTT_{FT}, and that of the value which is obtained by dividing the PWTT_{ET} by the ET. Therefore, the PWTT_{ET} and the PWTT_{FT}, the difference between the PWTT_{ET} and the PWTT_{FT}, and the value which is obtained by dividing the PWTT_{ET} by the ET are displayed in correlation with one another.

Although, in the display image illustrated in FIG. 9, the abscissa indicates the elapsed time from the timing of hospital discharge of the subject, the abscissa can indicate the acquisition time when the electrocardiogram, the earlobe pulse wave, and the pulse waves in the extremities are acquired. In this case, the ordinates can indicate the PWTT_{ET} and the PWTT_{FT} that are obtained from the electrocardiogram, earlobe pulse wave, and pulse waves in the extremities that are acquired at the acquisition time of the abscissa, the difference between the PWTT_{ET} and the PWTT_{FT}, and the value that is obtained by dividing the PWTT_{ET} by the ET. The acquisition time may be additionally indicated at each plot of the two-dimensional graphs illustrated in FIG. 9.

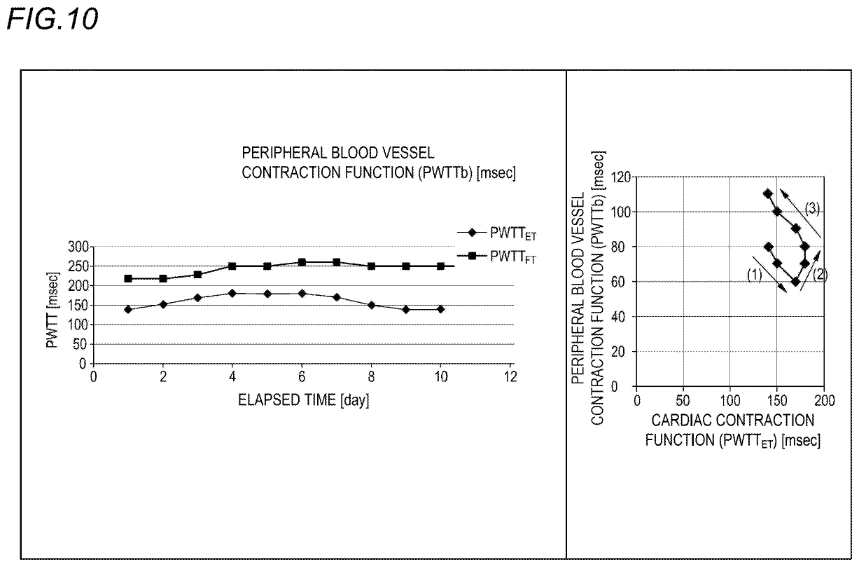

FIG. 10 illustrates an example of a display image in which two-dimensional graphs of relationships between the PWTT_{ET} and the PWTT_{FT}, and the PWTT_{ET} and the PWTTb (the difference between the PWTT_{ET} and the PWTT_{FT}) are simultaneously displayed. In the two-dimensional graph of the relationship between the PWTT_{ET} and the PWTTb, the change over time in the relationship between the PWTT_{ET} and the PWTTb is shown by the arrow indicating the direction of the change over time. In the two-dimensional graph, the PWTT_{ET} can be displayed as an index of the cardiac contraction function, and the PWTTb can be displayed as an index of the peripheral blood vessel contraction function indicating the state of the vascular resistance. According to the two-dimensional graph, it is possible to understand that the cardiac contraction function and the peripheral blood vessel contraction function track the following changes over time. In the step of (1) of the figure, namely, the cardiac contraction function is lowered, and the peripheral blood vessel dilates with the result that the blood pressure is lowered. In the step of (2), the lowered cardiac contraction function is compensated by the contraction of the peripheral blood vessel, and the blood pressure is maintained. In the step of (3), then, the cardiac function is recovered, and the peripheral blood vessel is further contracted, whereby the blood pressure is maintained.

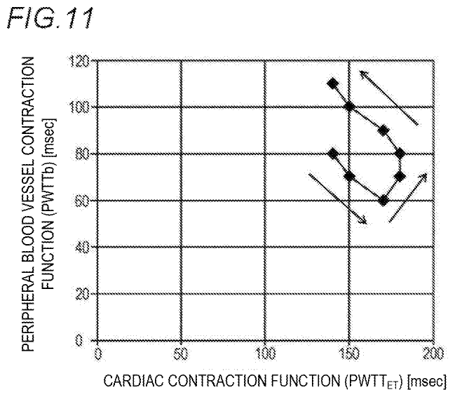

FIG. 11 illustrates an example of a display image of the two-dimensional graph of the relationship between the PWTT_{ET} and the PWTTb. In this way, only the two-dimensional graph of the relationship between the PWTT_{ET} and the PWTTb may be displayed as the display image.

Although, in FIGS. 10 and 11, the PWTT_{FT} is displayed as an index of the cardiac contraction function, the PWTT_{ET}/ET or the average values of the PWTT_{ET} and the PWTT_{ET}/ET may be displayed on the displaying section 160 in place of the PWTT_{ET}.

The embodiment attains the following effects.

The CPU 131 calculates information relating to the cardiac functions of the subject based on the first pulse wave transit time calculated from the electrocardiogram and the earlobe pulse wave (cranio-cervical pulse wave), and the second pulse wave transit time calculated from the electrocardiogram and one of the pulse waves in the extremities acquired from at least one of the extremities. That is, the apparatus 100A can compare the pulse wave transit time at a position that is proximal to the heart, and that at a position that is distal from the heart, with each other. As compared with the pulse wave of the upper arm, moreover, the earlobe pulse wave more correctly reflects the central information, and can be easily measured. Therefore, the apparatus 100A can simultaneously know the state of the cardiac contraction function of the subject, and that of the vascular resistance, and measure information relating to the cardiac contraction function of the subject, with a higher accuracy and with a light burden of the subject.

Moreover, the earlobe pulse wave that is the first pulse wave is acquired by the photoplethysmography sensor 121. In contrast to the brachial artery pulse wave that is acquired by performing a measurement while a cuff is attached to the upper arm of the subject, the acquisition of the earlobe pulse wave is enabled by causing the photoplethysmography sensor 121 to nip the earlobe. That is, the burden on the subject can be made smaller than the case where the pulse wave is measured by using a cuff. Moreover, the pulse wave of the subject can be continuously measured without restraining the motion of the subject as compared with the case where the pulse wave is measured by using a cuff.

According to the thus configured apparatus for measuring hemodynamic parameters, information relating to the cardiac functions of the subject is calculated based on: the first pulse wave transit time that is calculated from the electrocardiogram and the pulse wave of the cranio-cervical region; and the second pulse wave transit time that is calculated from the electrocardiogram and the pulse wave of the at least one of the extremities.

According to the configuration, therefore, the pulse wave transit time in the portion that is proximal to the heart, and that in the portion which is distal from the heart can be compared to each other, whereby the state of the cardiac contraction function of the subject, and that of the vascular resistance are enabled to be simultaneously known. Moreover, the pulse wave of the cranio-cervical region correctly reflects the central information. As compared with the measurement of the pulse wave of the upper arm by using a cuff, furthermore, the measurement of the pulse wave of the cranio-cervical region can be performed more simply. Therefore, the thus configured apparatus for measuring hemodynamic parameters can measure information relating to the cardiac contraction function of the subject, with a higher accuracy and with a light burden of the subject.

The presently disclosed subject matter is not limited to the above-described embodiments, and may be freely subjected to modifications, improvements, and the like. In addition, the materials, shapes, dimensions, values, forms, numbers, installation places, and the like of the components of the above-described embodiments are arbitrary and not limited insofar as the presently disclosed subject matter can be achieved.

Although, in the first and second embodiments, the apparatus for measuring hemodynamic parameters 100 and the apparatus for measuring hemodynamic parameters 100A include the displaying section 160 and the inputting section 170, at least one of the displaying section 160 and the inputting section 170 may be disposed in an external device that is different from the apparatus for measuring hemodynamic parameters 100. In this case, the controller 130 may be configured so as to be able to control the external device.

A part or all of the functions which are to be executed by programs in the embodiments may be executed by hardware such as electronic circuits.

What is claimed is:

1. An apparatus for measuring hemodynamic parameters comprising:
an electrocardiogram acquiring interface configured to acquire an electrocardiogram of a subject;
a pulse wave acquiring interface configured to acquire a pulse wave of a cranio-cervical region of the subject; and
a calculator configured to calculate information relating to cardiac functions of the subject, based on a pulse wave transit time obtained from the electrocardiogram and the pulse wave.

2. The apparatus for measuring hemodynamic parameters according to claim 1, wherein the pulse wave is acquired by a photoplethysmography sensor.

3. An apparatus for measuring hemodynamic parameters comprising:
an electrocardiogram acquiring interface configured to acquire an electrocardiogram of a subject;
a first pulse wave acquiring interface configured to acquire a first pulse wave in a cranio-cervical region of the subject;
a second pulse wave acquiring interface configured to acquire a second pulse wave in at least one of extremities of the subject;
a first calculator configured to calculate a first pulse wave transit time from the electrocardiogram and the first pulse wave; and
a second calculator configured to calculate a second pulse wave transit time from the electrocardiogram and the second pulse wave.

4. The apparatus for measuring hemodynamic parameters according to claim 3, wherein the first pulse wave is acquired by a photoplethysmography sensor.
